(19) United States
(12) Patent Application Publication
(10) Pub. No.: US 2005/0107702 A1
(43) Pub. Date: May 19, 2005

(54) External high-power focusing ultrasonic treatment

(76) Inventors: Shenxu He, Beijing (CN); Liulin Xiong, Beijing (CN); Jinsheng Yu, Beijing (CN)

Correspondence Address: LADAS & PARRY, 26 WEST 61ST STREET, NEW YORK, NY, 10023, US

(21) Appl. No.: 10/494,559

(22) PCT Filed: Mar. 15, 2002
(86) PCT No.: PCT/CN02/00168

(30) Foreign Application Priority Data
Nov. 5, 2001 (CN) 01134485.7

(51) Int. Cl. A61N 7/00

(52) U.S. Cl. 600/439; 601/3; 600/459

(57) ABSTRACT

The present invention provides an extracorporeal HIFU therapeutic apparatus including a HIFU source and a driving circuit thereof for providing HIFU; a locating system for searching the object to be treated and moving it to the focal point of the ultrasound transducer, which includes a medical imaging system, a carrying device for the patient, and a displacement system for causing the spatial movement of the carrying device with respect to the HIFU source; and a high intensity ultrasonic wave transmitter and a processing system for the transmission medium, the ultrasonic wave adapted to the HIFU being transmitted into the patient body through a transmission medium; a container for receiving the transmission medium in front of an emitting surface of the HIFU source and means for filling, discharging and processing the transmission medium, wherein the HIFU source is supported on a supporting means and is placed above the carrying device so as to form an upper-mounted ultrasonic wave source. The therapeutic apparatus can allow the patient to be treated in a supination pose so as to extend the treatment range.

### FIELD OF THE INVENTION

The present invention generally relates to an extracorporeal high intensity focus ultrasound (HIFU) therapeutic apparatus. More particularly, the invention relates to a medical apparatus for focusing a high intensity ultrasonic wave emitted from an extracorporeal ultrasonic wave source at a focal point so as to kill and damage the diseased tissue by the thermal effect of the high intensity focus ultrasonic wave.

### BACKGROUND OF THE DISCLOSURE

At present, studies on the HIFU therapeutic apparatus become popular in the field of medical study and clinical applications of HIFU have perfect therapeutic effect. The existing extracorporeal HIFU therapeutic apparatus generally includes the following means: a HIFU source and a driving circuit thereof for providing the HIFU; a locating system for searching the object to be treated and moving it to the focal point of the ultrasound transducer, which includes a medical imaging system (such as the B-Mode Ultrasonoscope), a carrying device for the patient such as a bed surface, and a displacement system for causing the spatial movement of the carrying device with respect to the ultrasonic wave source; a high intensity ultrasonic wave (HIU) transmitter and a processing system for transmission medium. Since the ultrasonic wave adapted to the HIFU must be transmitted into the patient body through a special transmission medium (such as deaerated water), a container for receiving the transmission medium in front of an emitting surface of the HIFU source (such as a water tank or a water bag etc.) and means for filling and discharging the transmission medium and processing them are necessary.

All the present extracorporeal HIFU therapeutic apparatus for clinical applications use the arrangement of a “lower-mounted ultrasonic wave source”, that is, the HIFU source is positioned below the bed surface of the therapeutic apparatus. The ultrasonic wave is transmitted into a patient body through the deaerated water contained in an open-type water tank opening into the therapy bed surface. The HIFU therapeutic apparatus with the lower-mounted ultrasonic wave source has the following disadvantages: when treating the tumors in the abdominal cavity and pelvic cavity by HIFU, for the most of patient, only from the front can the ultrasonic wave be transmitted into the body because the ultrasonic wave may be blocked by the spine and the pelvis when transmitted from the back of the patient. Therefore, in this case, during treatment, most of patients shall be only in a pronation pose when being treated by the HIFU therapeutic apparatus with the lower-mounted ultrasonic wave source. The disadvantages due to the pronation pose are as follows: 1. the physical strength dissipation of the patient will be much more and the patient can not bear a long time treatment; 2. when the patient is in the pronation pose, the abdominal wall and the intestinal canals will sag under the gravity action while the positions of the tumors are relative fixed, which cause the distance from the abdominal wall to the tumors to substantially increase so as to cause the increase of the ultrasound attenuation along the transmission path of the HIFU, and the ultrasound attenuation will reduce the sound intensity at focal point so as to affect the therapy effect; 3. the gas in the stomach and intestine will disturb the locating of B-Mode Ultrasonoscope. In addition, in the case of the lower-mounted wave source, since the therapy cavity is open and shall be filled with deaerated water, the range of the inclining angle of the ultrasonic wave source is so narrow that the tumors behind the costal margin, xiphisternum and pubis can not be treated.

### SUMMARY OF THE INVENTION

The object of the present invention is to overcome the above-mentioned disadvantages by providing an extracorporeal HIFU therapeutic apparatus in which the HIFU source can be placed above the bed surface so as to allow the patient to be treated in a supination pose so that the physical strength dissipation of the patient will be substantially reduced and the tumors behind the costal margin, xiphisternum and pubis may be treated, which extends the treatment range of the HIFU therapeutic apparatus.

In order to fulfill the above-mentioned object, the present invention provides an extracorporeal HIFU therapeutic apparatus including a HIFU source and a driving circuit thereof for providing HIFU; a locating system for searching the object to be treated and moving it to the focal point of the ultrasound transducer, which includes a medical imaging system, a carrying device for carrying the patient, and a displacement system for causing the spatial movement of the carrying device with respect to the HIFU source; and a high intensity ultrasonic wave (HIU) transmitter and a processing system for the transmission medium in which the ultrasonic wave adapted to the HIFU is transmitted into the patient body through the transmission medium; a container for receiving the transmission medium in front of an emitting surface of the HIFU source and means for filling, discharging and processing the transmission medium, wherein the HIFU source is supported on a supporting means and is placed above the carrying device for patient so as to form an upper-mounted ultrasonic wave source.

The apparatus of the present invention can further includes the following features:

The container for the transmission medium may be a water bag. The displacement system may include the following features: the upper-mounted ultrasonic wave source is rotatably mounted on the ultrasonic wave source supporting means and the ultrasonic wave source supporting means can be moved up and down so that the upper-mounted ultrasonic wave source can be rotated around the axes X and Y respectively and moved up and down along the axis Z; the carrying device for patient is provided with a screw-nut arrangement so that it can move in a transverse direction S and a longitudinal direction L, and the carrying device is also provided with a gear mechanism so that it can be rotated around the longitudinal axis L.

In addition, the extracorporeal HIFU therapeutic apparatus according to the present invention can further be provided with a lower-mounted ultrasonic wave source which is positioned below the carrying device for patient, and a containing means for the transmission medium is provided above the lower-mounted ultrasonic wave source. A part of the carrying device facing the lower-mounted ultrasonic wave source is detachable, and it further includes a seat frame on which the patient may sit during treatment. The lower-mounted ultrasonic wave source is also rotatable around the axes X and Y respectively and can move up and down along the axis Z.

According to the present invention, with the upper-mounted ultrasonic wave source, the patient can be treated in a supination pose so that the physical strength dissipation of the patient will be substantially reduced. Meanwhile, when the patient is treated in a supination pose, under the gravity action and the compression force of the water bag, the distance from the skin to the tumors may be substantially reduced and the gas in the intestine will be extruded, and thus the therapy effect on the tumors in abdomen, especially on the adenocarcinoma of pancreas and the lymphnode metastatic carcinoma in abdomen will be greatly improved. Further, the upper-mounted ultrasonic wave source can be freely rotated and inclined so that the tumors behind the costal margin, xiphisternum and pubis can be treated, which extends the treatment range of the HIFU therapeutic apparatus

### BRIEF DESCRIPTION OF DRAWINGS

The present invention will be described in detail by the preferred embodiments with reference to the following drawings, wherein,

FIG. 1 is a structural schematic view of an embodiment of the HIFU therapeutic apparatus according to the present invention;

FIG. 2 is a schematic view showing the operational principle of the embodiment shown in the FIG. 1 when performing the treatment;

FIG. 3 is a structural schematic view of another embodiment according to the present invention;

FIG. 4 is a schematic view showing the operational principle of the embodiment shown in FIG. 3;

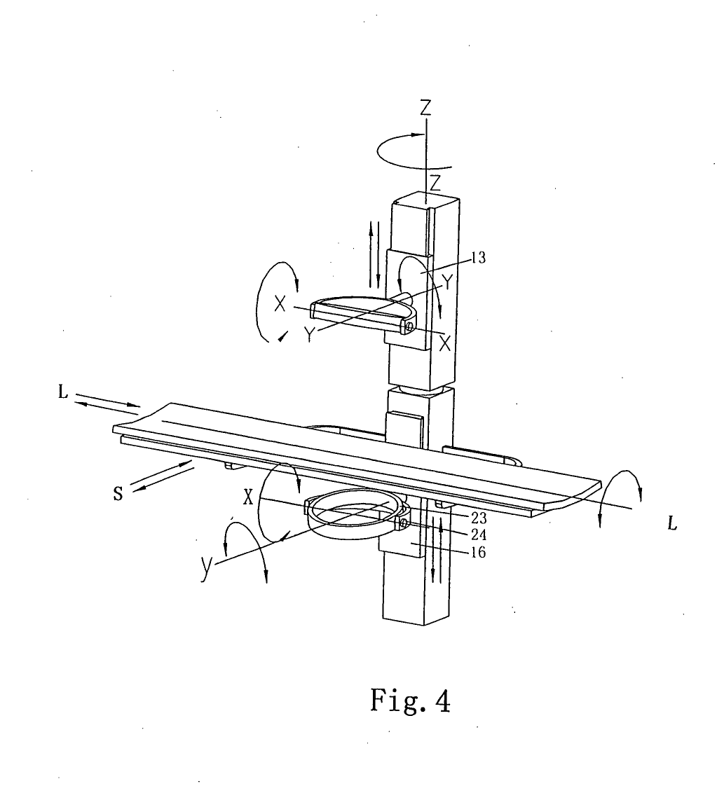

FIG. 5 is a structural schematic view showing the carrying device for patient of FIG. 1 or 3 moving along its longitudinal direction L;

FIG. 6 is a structural schematic view showing the carrying device for patient of FIG. 1 or 3 moving along its transverse direction S and turning around the longitudinal direction L;

### THE BEST MODE OF THE EMBODIMENTS

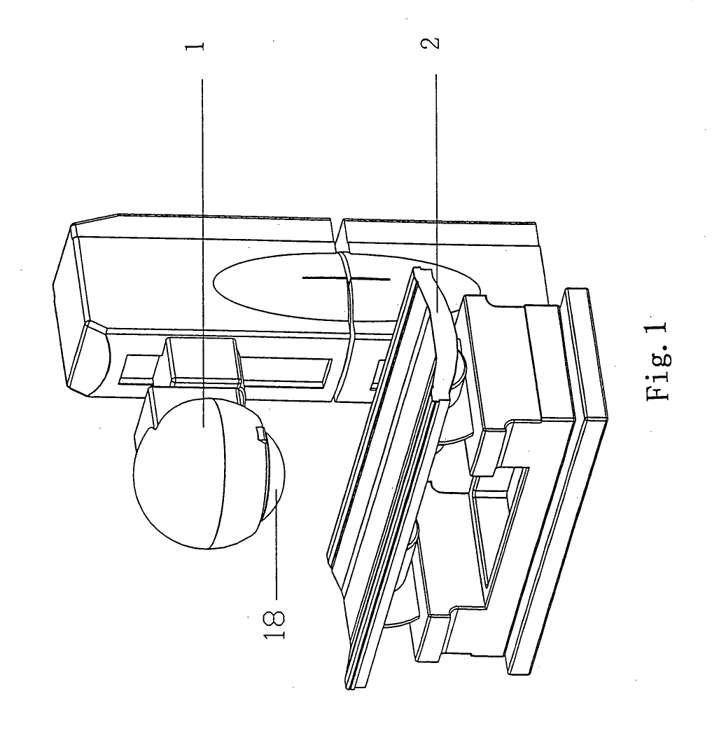

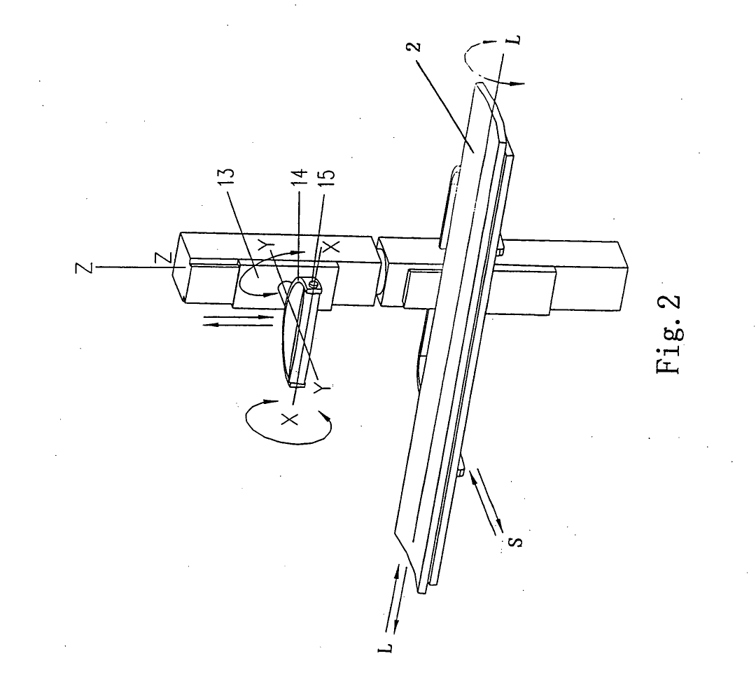

With reference to FIG. 1, the present invention provides an extracorporeal HIFU therapeutic apparatus, in which the reference number 1 designates an upper-mounted ultrasonic wave source, the reference number 2 designates the carrying device for patient which can be a bed surface. FIG. 2 shows a schematic view showing the movement of the relevant parts in the embodiment, i.e. it shows an operational principle view which does not include the telescoping movement and rotation of the probe head of the B-Mode Ultrasonoscope known in the prior art.

As seen in FIG. 1, the HIFU therapeutic apparatus in the embodiment includes: a HIFU source 1 and a driving circuit thereof for generating HIFU; a locating system for searching the object to be treated and moving it to the focal point of the ultrasound transducer which includes a medical imaging system (not shown) known in the prior art, a carrying device 2 for carrying the patient, and a displacement system for causing the spatial movement of the carrying device 2 for patient with respect to the HIFU source 1; and a high intensity ultrasonic wave (HIFU) transmitter and a processing system for the transmission medium (not shown, which is known the prior art), in which the ultrasonic wave adapted to the HIFU is transmitted into the patient body through the transmission medium; a container for receiving the transmission medium in front of the emitting surface of the HIFU source 1 and means for filling, discharging and processing the transmission medium (not shown, which are known the prior art), wherein the HIFU source 1 is supported on a supporting means 13 and is placed above the carrying device 2 for patient so as to form an upper-mounted ultrasonic wave source.

The medical imaging system may be a B-Mode Ultrasonoscope; the transmission medium may be deaerated water; and the container for the transmission medium may be a water bag 18.

In the embodiment shown in FIG. 2, the displacement system includes the following features: the upper-mounted ultrasonic wave source 1 is rotatably mounted on the ultrasonic wave source supporting means 13 and the ultrasonic wave source supporting means 13 can be moved up and down so that the upper-mounted ultrasonic wave source 1 can be rotated around the axes X and Y respectively and moved up and down along the axis Z. Specifically, the upper-mounted ultrasonic wave source 1 is horizontally supported on the ultrasonic wave source supporting means 13 placed vertically by a supporting frame 14 and can be rotated around an axis Y, the supporting frame 14 is provided with a pivot shaft 15 which can be rotated around an axis X so that the upper-mounted ultrasonic wave source 1 may be rotated around the axis X by means the pivot shaft 15; the ultrasonic wave source supporting means 13 is a screw-nut arrangement (not shown, which is known in the prior art) so that the upper-mounted ultrasonic wave source 1 can move up and down along an axis Z.

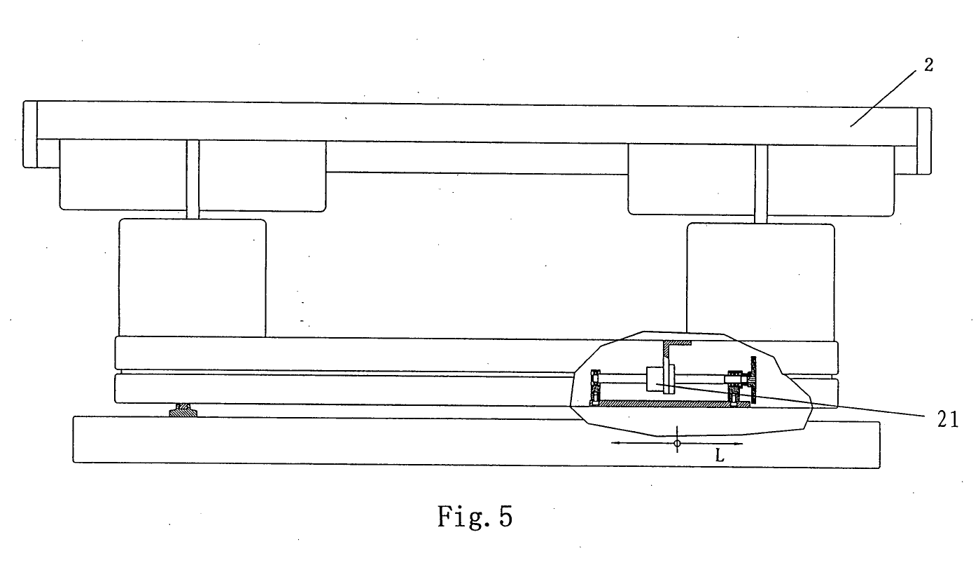

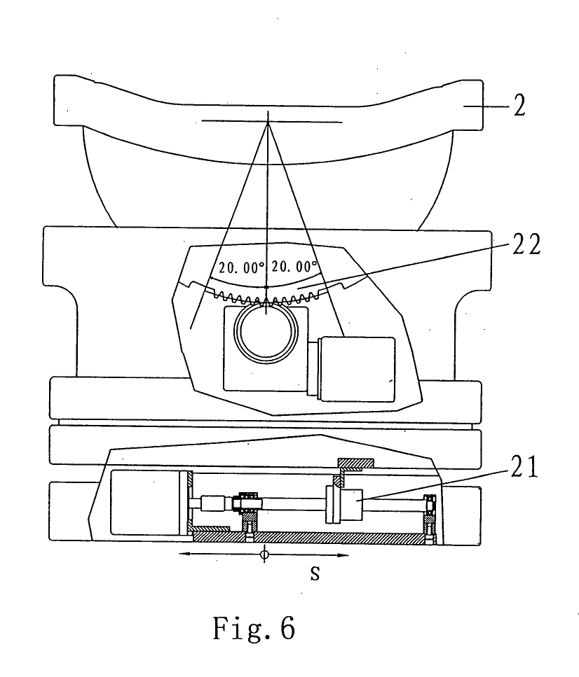

In addition, in the displacement system, the carrying device 2 for patient is provided with a screw-nut arrangement 21 so that it can move in a transverse direction S and a longitudinal direction L, and the carrying device is provided with a gear mechanism 22 so that it can be rotated around the longitudinal axis L, and the rotation angle can be 40°, as shown FIGS. 5 and 6, which is known in the prior art.

When being treated, the patient may lie on the carrying device 2 in a supination pose, the upper-mounted ultrasonic wave source 1 can be rotated and moved up and down so as to be adjusted to a desired angle, meanwhile, the carrying device 2 can be moved in a transverse direction S and a longitudinal direction L or rotated about the longitudinal L to a desired position and angle. Therefore, the HIFU therapeutic apparatus according to the present invention overcomes the disadvantages of the conventional HIFU therapeutic apparatus that the patient can be only in a pronation pose when being treated.

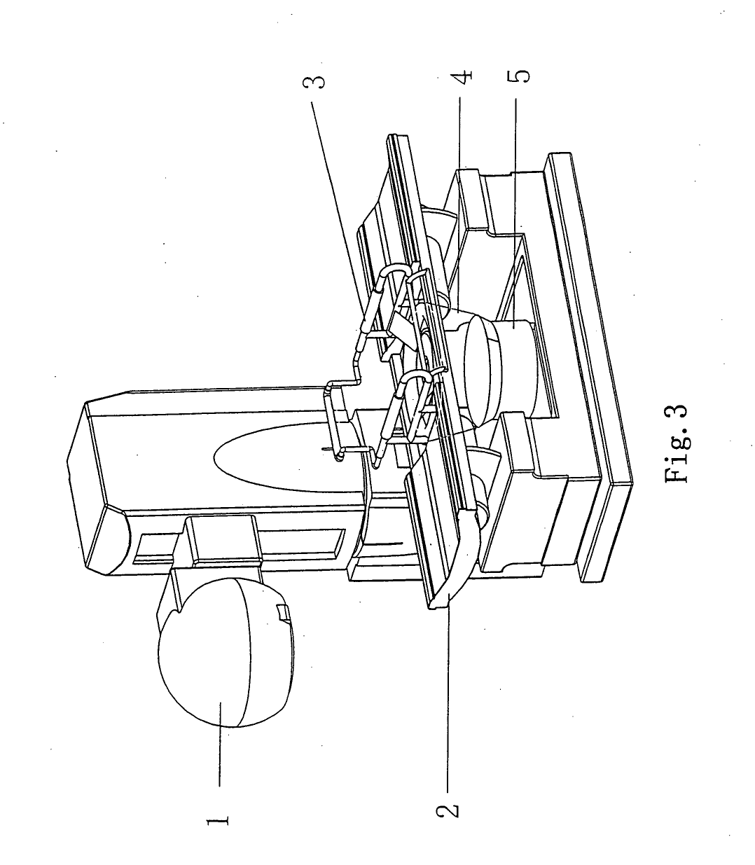

FIG. 3 shows a second embodiment of the present invention, in which the upper-mounted ultrasonic wave source 1 and a lower-mounted ultrasonic wave source 5 of a chair type are combined. In addition to the upper-mounted ultrasonic wave source 1, the extracorporeal HIFU therapeutic apparatus further includes a lower-mounted ultrasonic wave source 5 which is positioned below the carrying device 2 for patient, a container for the transmission medium is provided above the lower-mounted ultrasonic wave source 5, and a part of the carrying device 2 facing the lower-mounted ultrasonic wave source is detachable.

The container for the transmission medium may be an open-type water tank 4.

The apparatus in the embodiment further includes a seat frame 3 on which the patient may sit when being treated.

In the displacement system in this embodiment, like the upper-mounted ultrasonic wave source 1, the lower-mounted ultrasonic wave source 5 may be rotatably mounted on the ultrasonic wave source supporting means 13 so that the lower-mounted ultrasonic wave source 5 can be rotated around the axes X and Y respectively and moved up and down along the axis Z. The ultrasonic wave source supporting means 13 may be configured as a sleeve rotatable around an axis Z so that the upper-mounted ultrasonic wave source 1 is rotatable around the axis Z.

In the embodiment, when the patient is treated by using the upper-mounted ultrasonic wave source, like the embodiment of FIG. 1, the patient can lie on the bed surface. When treated by using the lower-mounted ultrasonic wave source, the upper-mounted ultrasonic wave source 1 shall be rotated about 90° and the detachable portion of the bed surface in the middle of the carrying device (therapy bed) 2 is removed and replaced by the seat frame 3. When the patient sits on the seat frame 3, the ultrasonic wave will be transmitted into the body through the perineum so as to perform the treatment.

The ultrasonic wave source driving circuit is known in the prior art.

According to the present invention, with the upper-mounted ultrasonic wave source, the following beneficial effects can be obtained: 1. the patient with tumors in the abdominal cavity and pelvic cavity can be treated in an upination pose so that the physical strength dissipation of the patient will be substantially reduced; 2. when the patient being treated in a supination pose, the distance from the skin to the tumors may be substantially reduced under the gravity action and compression force of the water bag while the gas in the intestine will be extruded, and thus the therapy effect on the tumors in abdomen, especially on the adenocarcinoma of pancreas and the lymphnode metastatic carcinoma in abdomen will be greatly improved; 3. the upper-mounted ultrasonic wave source can be freely rotated and inclined so that he tumors behind the costal margin, xiphisternum and pubis can be treated, which extends the treatment range of the HIFU therapeutic apparatus; 4. reduction of the distance from the skin to the tumors may reduce the disturbing of the gas so that realtime monitoring of the B-Mode Ultrasonoscope will be clearer during treatment.

Alternatively, according to the present invention, the open-type water tank can be replaced by a closed water bag to transmit the ultrasonic wave. Thus, the patient skin will not contact the deaerated water directly so that HIFU therapy becomes a “dry” therapy.

1. An extracorporeal HIFU therapeutic apparatus including: a HIFU source (1) and a driving circuit thereof for generating HIFU; a locating system for searching the object to be treated and moving it to the focal point of the ultrasound transducer, which includes a medical imaging system, a carrying device (2) for carrying the patient, and a displacement system for causing the spatial movement of the carrying device (2) for patient with respect to the HIFU source (1); a high intensity ultrasonic wave transmitter and a processing system for the transmission medium, the ultrasonic wave adapted to the HIFU being transmitted into the patient body through the transmission medium; a container for receiving the transmission medium in front of an emitting surface of the HIFU source (1) and means for filling, discharging and processing the transmission medium, wherein the HIFU source (1) is supported on a ultrasonic wave source supporting means (13) and is placed above the carrying device (2) for patient so as to form an upper-mounted ultrasonic wave source.

2. An extracorporeal HIFU therapeutic apparatus according to claim 1, wherein the medical imaging system is a B-Mode Ultrasonoscope.

3. An extracorporeal HIFU therapeutic apparatus according to claim 1, wherein the transmission medium is deaerated water.

4. An extracorporeal HIFU therapeutic apparatus according to claim 1, wherein the container for the transmission medium is a water bag (18).

5. An extracorporeal HIFU therapeutic apparatus according to claim 1, wherein the displacement system is configured in such a manner that the upper-mounted ultrasonic wave source (1) is rotatably mounted on the ultrasonic wave source supporting means (13) and the ultrasonic wave source supporting means (13) can be moved up and down so that the upper-mounted ultrasonic wave source (1) can be rotated around the axes X and Y respectively and moved up and down along the axis Z.

6. An extracorporeal HIFU therapeutic apparatus according to claim 5, wherein the upper-mounted ultrasonic wave source (1) is rotatably mounted on the ultrasonic wave source supporting means (13) in such a manner that the upper-mounted ultrasonic wave source (1) is horizontally supported on the vertical ultrasonic wave source supporting means (13) by a supporting frame (14) and is rotatable around an axis Y, and the supporting frame (14) is provided with a pivot shaft (15) which is rotatable around an axis X, the upper-mounted ultrasonic wave source (1) being rotatable around the axis X by means of the pivot shaft; and the ultrasonic wave source supporting means (13) is a screw-nut arrangement so that the upper-mounted ultrasonic wave source (1) is movable up and down along an axis Z.

7. An extracorporeal HIFU therapeutic apparatus according to claim 1, wherein in the displacement system, the carrying device (2) for patient is provided with a screw-nut arrangement (21) so that it can move in a transverse direction (S) and a longitudinal direction (L), and the carrying device is provided with a gear mechanism (22) so that it can be rotated around the longitudinal axis L.

8. An extracorporeal HIFU therapeutic apparatus according to claim 1, wherein the extracorporeal HIFU therapeutic apparatus further includes a lower-mounted ultrasonic wave source (5) which is positioned below the carrying device (2) for patient, a container for the transmission medium is provided above the lower-mounted ultrasonic wave source (5), a part of the carrying device 2 facing the lower-mounted ultrasonic wave source (5) is detachable.

9. An extracorporeal HIFU therapeutic apparatus according to claim 8, wherein the container for the transmission medium is an open-type water tank (4).

10. An extracorporeal HIFU therapeutic apparatus according to claim 8, further including a seat frame (3) on which the patient may sit when being treated.

11. An extracorporeal HIFU therapeutic apparatus according to claim 8, wherein in the displacement system, the ultrasonic wave source supporting means (13) is configured as a sleeve rotatable around an axis Z so that the upper-mounted ultrasonic wave source (1) is rotatable around the axis Z.

12. An extracorporeal HIFU therapeutic apparatus according to claim 8, wherein in the displacement system, a lower-mounted ultrasonic wave source (5) is rotatably mounted on the ultrasonic wave source supporting means (13) which is movable up and down so that the lower-mounted ultrasonic wave source (5) is rotatable around the axes X and Y respectively and movable up and down along the axis Z.

13. An extracorporeal HIFU therapeutic apparatus according to claims 12, wherein the lower-mounted ultrasonic wave source (5) is rotatably mounted on the ultrasonic wave source supporting means (13) in such a manner that the lower-mounted ultrasonic wave source (5) is horizontally supported on the vertical ultrasonic wave source supporting means (13) by a supporting frame (13) and is rotatable around an axis Y, and the supporting frame (23) is provided with a pivot shaft (24) rotatable around an axis X, the lower-mounted ultrasonic wave source (5) being rotatable around the axis X by means the pivot shaft (24); and the ultrasonic wave source supporting means (13) is a screw-nut arrangement so that the lower-mounted ultrasonic wave source (5) can move up and down along an axis Z.
